(19) United States
(12) Patent Application Publication
(10) Pub. No.: US 2009/0311266 A1
(43) Pub. Date: Dec. 17, 2009

(54) Inhibitors of the endothelin signalling pathway and alphavbeta3 integrin receptor antagonists for combination therapy

(75) Inventors: Wilhelm Amberg, Mannheim (DE); Andreas Kling, Mannheim (DE); Wilfried Hornberger, Neustadt (DE)

Correspondence Address: POLSINELLI SHUGHART PC, 700 W. 47TH STREET, SUITE 1000, KANSAS CITY, MO, 64112-1802, US

(73) Assignee: BASF AKTIENGESELLSCHAFT, Ludwigshafen (DE)

(21) Appl. No.: 12/433,295

(22) Filed: Apr. 30, 2009

Related U.S. Application Data
(63) Continuation of application No. 10/089,146, filed on Sep. 16, 2002, now abandoned.

(30) Foreign Application Priority Data
Oct. 6, 1999 (DE) 199482691
Dec. 24, 1999 (DE) 199629986
Jun. 6, 2000 (DE) 100275141
Jun. 14, 2000 (DE) 100285759
Aug. 11, 2000 (DE) 100399983

(51) Int. Cl. A61K 39/395

(52) U.S. Cl. 424/143.1

(57) ABSTRACT

The invention relates to the use of an endothelin blocker in combination with an αᵥβ₃ integrin receptor antagonist for the treatment or prevention of diseases, particularly to the use of a pharmaceutical composition, comprising an endothelin blocker and an αᵥβ₃ integrin receptor antagonist, for the treatment or prevention of cardiovascular disorders, particularly for the treatment or prevention of restenosis after vessel injury or revascularisation treatment and to the pharmaceutical composition itself.

The invention relates to the use of an endothelin blocker in combination with an αᵥβ₃ integrin receptor antagonist for the treatment or prevention of diseases, particularly to the use of a pharmaceutical composition, comprising an endothelin blocker and an αᵥβ₃ integrin receptor antagonist, for the treatment or prevention of cardiovascular disorders, particularly for the treatment or prevention of restenosis after vessel injury or revascularisation treatment and to the pharmaceutical composition itself.

Percutaneous transluminal coronary angioplasty (PTCA) was first introduced into the therapy of patients with coronary artery stenosis in the late seventies. In the two decades since this method has become the standard therapy for patients suffering from all forms of coronary artery disease. The success rate of the procedure itself has increased from 61 percent in the late seventies to well over 90 percent from the mid-eighties onwards. However, long-term success of PTCA remains limited by late restenosis caused by vessel wall proliferation that occurs in 20 to 40 percent of all patients to such an extent that a second PTCA is necessary [Anderson V H, Smalling R W, Serruys P W. Mechanical devices. In: Willerson J T, Cohn J N (eds). Cardiovascular Medicine. New York, Edinburgh, London: Churchill Livingstone, 1995:617-651].

As in the mid-90s about 500,000 primary PTCA procedures were carried out in the USA and about 200,000 in Europe, this accounts for more than 230,000 patients per year eligible for a second invasive procedure due to recurrent angina. A hypothetical reduction of the incidence of restenosis by 10-15 percent per year would reduce annual treatment costs by almost 1.5 billion dollars in the USA and Europe alone.

Up to now a large number of clinical trials performed to investigate whether systemic administration of drugs which were effective in animal models of restenosis were efficacious in men have failed. The reason for this discrepancy between pre-clinical and clinical studies could be that doses effective in experimental settings have not been applicable to patients due to other cardiovascular effects of these drugs (Pratt R E, Dzau V E. Pharmacological strategies to prevent restenosis. Circulation 1996; 93:848-852].

Meanwhile revascularisation by balloon dilatation, stentimplantation, laser or rotablator are used not only in coronary arteries, but in all other large arteries accessible if atherosclerotic lessions make such an intervention necessary (Stenosis of renal arteries, carotid arteries, femoral and brachial arteries). Thus the number of patients having problems with restenosis after any such intervention steadily increases posing a huge therapeutical and economic (reduction in health care costs) potential for drugs effectively inhibiting restenosis.

Endothelin (ET), a 21 amino acid peptide, has been described as the most potent endogenous vasoconstrictor known. [Yanagisawa M, Kurihara H, Kimura S, et al. A novel potent vasoconstrictor peptide produced by vascular endothelial cells. Nature 1988; 332:411-415]. Its precursor is big-Endothelin, which is cleaved to ET by ECE (endothelin converting enzyme). ET binds in an autocrine/paracrine fashion to two different specific high affinity receptors, named ET_{A} and ET_{B}. Within the vasculature ET_{A} receptors are only located on smooth muscle cells (SMCa) leading to vasoconstriction and SMC proliferation. In addition, a variable portion of ET_{B} receptors were also described on SMCs promoting at that location the same effect as ET_{A} receptors via the same intracellular signalling pathways [Rubanyi G M, Polokoff M A. Endothelins: Molecular biology, biochemistry, pharmacology, physiology, and pathophysiology. Pharmacol Rev 1994; 46:325-415].

It is known, that ET_{A} receptor antagonists effect on restenosis. The selective ET_{A} receptor antagonist A 127722 was tested in pigs with coronary artery stents [McKenna C J, Burke S E, Opgenorth T J, et al., Selective ET_{A} receptor antagonism reduces neointimal hyperplasia in a porcine coronary stent model, Circulation 1998, 97, 2551-2556]. After 28 days of oral treatment (b.i.d.) a maximal reduction in neointima formation of about 30% has been reported.

Arterial injury after revascularisation also triggers the expression of vascular membrane-bound protein heterodimers called integrins especially of integrins αᵥβ₃ and αᵥβ₅ [Corjay M H, Diamond S M, Schlingmann X L, Gibbs S K, Stoltenborg J K, Racanelli A L. AlphaVbeta3, alphaVbeta5, and osteoponfin are coordinately upregulated at early time points in a rabbit model of neointima formation. J Cell Biochem 1999; 75:492-504] and it is known since 1994 that formation of neointimal hyperplasia as the leading cause of restenosis can be inhibited by antagonists to integrin αᵥβ₃ [Choi E T, Engel L, Callow A D, Sun S, Trachtenberg J, Santoro S, Ryan U S. Inhibition of neointimal hyperplasia by blocking αᵥβ₃ integrin with a small peptide antagonist GpenGRGDSPCA J Vasc Surg 1994; 19:125-134].

First experimental hints came from the work of Sriramarao and co-workers which could show that endothelial attachment and spreading as basic mechanisms of angiogenesis is mediated by integrins αᵥβ₁ and αᵥβ₃ and could be inhibited by the integrin αᵥβ₃ specific antibody LM-609 and by RGD-containing peptides [Sriramarao P, Mendler M, Bourdon M Endothelial cell attachment and spreading on human tenascin is mediated by alpha 2 beta 1 and alpha v beta 3 integrins. J Cell Science 1993; 105:1001-1012]. Based on the clinical success of the anti-platelet integrin α_{IIb}β₃ antibody abciximab (c7E3 Fab, Reopro®) which led to sustained suppression of ischemic complications (endpoints death, MI or repeat intervention) after coronary interventions [Topol E J, Ferguson J J, Weisman H F, Tcheng J E, Ellis S G, Kleiman N S, Ivanhoe R J, Wang A L, Miller D P, Anderson K M, Califf R M. Long-term protection from myocardial ischemic events in a randomized trial of brief integrin beta3 blockade percutaneous coronary intervention. EPIC Investigator Group. Evaluation of Platelet IIb/IIIa Inhibition for Prevention of Ischemic Complication. JAMA 1997; 278:479-84] and the finding that it has similar affinity to both integrin α_{IIb}β₃ and αᵥβ₃ [Tam S S, Sassoli P M, Jordan R E, Nakada M T. Abciximab (ReoPro, chimeric 7E3 Fab) demonstrates equivalent affinity and functional blockade of glycoprotein IIb/IIIa and alpha(v)beta3 integrins. Circulation 1998; 98:1085-1091] it was suspected that besides acute thrombosis abciximab was also able to prevent vascular restenosis.

This anti-proliferative effect could be demonstrated in a porcine coronary injury model, where a selective integrin αᵥβ₃ blockade by the peptidic compound XJ 735 potently reduced neointimal hyperplasia by 43% and led to 2.9 fold less lumen stenosis [Srivatsa S S, Fitzpatrick L A, Tsao P W, Reilly T M, Holmes D R Jr, Schwartz R S, Mousa S A. Selective alpha v beta 3 integrin blockade potently limits neointimal hyperplasia and lumen stenosis following coronary arterial stent injury: evidence for the functional importance of integrin alpha v beta 3 and osteopontin expression during neointima formation. Cardiovasc Res. 1997; 36:408-28].

In addition integrin αᵥβ₃ antagonists have been shown to themselves exhibit some antithrombotic activity due to inhibition of αᵥβ₃ mediated platelet function like adhesion to the vessel wall [Gawaz M, Neumann F J, Dickfeld T, Reininge A, Adelsberger H, Gebhardt A, Schomig A. Vitronectin receptor (alpha(v)beta3) mediates platelet adhesion to the luminal aspect of endothelial cells: implications for reperfusion in acute myocardial infarction. Circulation. 1997; 96:1809-18].

Both receptors, namely ET receptors and integrin αᵥβ₃, play a role in restenosis after vascular injury both in animal models and in man. By inhibiting either of the two principles, a 30 to 40 percent reduction of neointima formation could be achieved in the relevant experimental models.

It is an object of the present invention to provide an effective method of treatment or prevention of cardiovascular disorders, particularly of restenosis after revascularisation, with acceptable side effects and advantageous properties.

We have found that this object is achieved by using an endothelin blocker in combination with an αᵥβ₃ integrin receptor antagonist.

By combining compounds which act as ET blockers and αᵥβ₃ integrin receptor antagonist either in one formulation or as a kit-of-parts combination by applying both separately via the same or different routes, it is possible to achieve a reduction of restenosis significantly more pronounced than one of the two treatments alone at the given doses. The combination of an ET blocker and an αᵥβ₃ integrin receptor antagonist in doses too low to be effective alone is as least as effective as a high mono-therapy with either agent and has less potential for side-effects than one principle alone.

Therefore, the invention relates to the use of an endothelin blocker in combination with an αᵥβ₃ integrin receptor antagonist for the manufacture of medicaments for the treatment or prevention of diseases, particularly of cardiovascular disorders, particularly of restenosis after vessel injury or revascularisation treatment.

Cardiovascular disorders are, for example, atherosclerosis, restenosis after vessel injury or revascularisation treatment, angioplasty (neointima formation, smooth muscle cell migration and proliferation), myokard infarkt or heart failure. In a preferred embodiment, the combination according to the invention can be used for the manufacture of medicaments for the treatment or prevention of restenosis after vessel injury or revascularisation treatment.

According to the invention, restenosis preferred means the sum of angiographic end points and clinical events, i.e. late (30 days after intervention and later) proliferative thickening of the wall and loss of minimal lumen diameter (MLD) of the vessel subjected to angioplasty together with the clinical endpoints death, myocardial infarction and repeat intervention (PTCA).

Restenosis can occur after vessel injury or revascularisation treatment. Revascularisation treatment preferred means methods of percutaneous transluminal angioplasty (PTA), such as balloon dilatation, stentimplantation, laser or rotablator. These methods are used not only in coronary arteries (percutaneous transluminal coronary angioplasty (PTCA)), but also in other large arteries accessible if atherosclerotic lessions make such an intervention necessary (Stenosis of renal arteries, carotid arteries, femoral and brachial arteries).

According to the invention, endothelin blacker means an inhibitor of the Endothelin Signalling Pathway such as, for example, endothelin receptor antagonists (ET antagonist), ECE inhibitors, antibodies against ET or ECE or modulators of expression of ET-precursor proteins or ET-receptors, particularly inhibitors of the Big-ET expression. Preferred endothelin blockers are ET antagonists, ECE inhibitors, or Antibodies against ET or ECE, most preferred endothelin blockers are ET antagonist. Preferred ECE Inhibitors within the scope of the invention are compounds which have a Kᵢ value of 1 μM or less. Most preferred are those ECE Inhibitors which have a Ki value of 100 NM or less and mostly preferred are those ECE Inhibitors which have a Ki value of 10 nM or less.

Suitable for the combination therapy of the invention are in principle all ECE inhibitors, for example peptidic and non-peptidic inhibitors, preferred are non-peptidic inhibitors, more preferred such which are orally available, such as Pohosphoramidon; CGS-31447: 1-{[(1S)-2-[1,1′-biphehyl]-4-yl-1-(1H-tetraazol-5-yl)ethyl]aminol-3-(1-naphthyl)propylphosphonic acid; CGS-34043: {[(1S)-2-dibenzor[b,d]furan-3-yl-1-(1H-tetraazol-5-yl)ethyl]amino}methylphosphonic acid; CGS-35066: (2S)-3 dibenzo[b,d]furan-3-yl-2-[(phosphonome-thyl)amino] propanoic acid; CGS-35339: (2S)-3-dibenzo[b,d]furan-3-yl-2-{[(diphenoxyphosphoryl)methyl] amino}propanoic acid; CGS-35066; WS-79089A: 1,6,9,14-tetrahydroxy-3-(2-hydroxypropyl)-7-methoxy-8,13-dioxo-5,6,8,13-tetrahydrobenzo[a]naphtha-cene-2-carboxylic acid; WS-75624A: (6-[2-(6-hydroxyheptyl)-1,3thiazol-4-yl]-4,5-dimethoxy-2-pyridinecarboxylic acid); PD-069185: N-(1-ethyl-3-piperidinyl)-6-iodo-2-(trichloromethyl)-4-quinazolinamine; SCH-54470: N-(1-(Hydroxy(l(R)—(N-alpha-(methylsulfonyl)-L-lysylamino)-2-phenylethyl) phosphinylmethyl)cyclopentylcarbonyl)-L-tryptophan dilithium salt; RU-69296: (2S)-2-{[2-(3-bromobenzyl)-3-sulfanylpropanoyl]amino}-3-(1H-indol-3-yl) propanoic acid; RU-69739: (2S)-2-([2-(4-bromobenzyl)-3-sulfanylpropanoyl]amino}-3-(1H-indol-3-yl) propanoic acid; KC-12792-2-AB; SLV-306: ((35,2′R)-3-(1-(2′-(Ethoxycarbonyl)-4′-phenyl-butyl-)-cyclopentan-1-carbonylamino)-2,3,4,5-tetrahydro-2-oxo-1H-benzapin-1-acetic acid; FR-901533; Preferred Endothelin receptor antagonists within the scope of the invention are substances which have a Kᵢ value of 1 μLM or less for either the ET_{A} receptor or the ET_{B} receptor or for both receptors at the same time. Most Preferred are those endothelin receptor antagonists which have a Kᵢ value of 100 nM or less and mostly preferred are those endothelin receptor antagonists which have a Kᵢ value of 10 nM or less. The Kᵢ value of endothelin receptor antagonists can be measured as described in DE 19636046 A1.

Suitable endothelin receptor antagonists for the combination therapy of the invention are in principle all endothelin receptor antagonists, peptidic and non-peptidic antagonists, for example as described in WO 96/22978, WO 98/27070, WO 98/09953, EP 617001, WO 98/22482, WO 97/30045, WO 9963936, WO 9833780, WO 9854116, WO9842709, WO 9841521, WO 9849162, WO 9717342, WO 9813366, WO 9739000, WO 9730045, WO 2000001389, WO 9937639, WO 9912916, WO 05132, WO 9728154, WO 9612706, WO 9827091, DE 19612101, DE 19609597, U.S. Pat. No. 5,716,984, U.S. Pat. No. 5,939,446, U.S. Pat. No. 5,922,681, U.S. Pat. No. 6,048,893 or GB 2337048, preferred are non-peptidic antagonists, more preferred such which are orally available.

Examples for peptidic endothelin receptor antagonists are:

FR-139317 (Perhydroazepine-1-ylcarbonyl-L-leucyl-(1-methyl)-D-tryptophyl-[3-(2-pyridyl)]-D-alanine); FR-901367 (2-Acetamido-3-[[1,4,4a,5,6,6a,7,12,12a,12b-decahydro-4a,8,12a,12b-tetrahydroxy-3-methyl-1,7,12-trioxobenz[a]anthracene-6a-yl]thio]propionic acid); BE-18257B (Cyclo(-D-Trp-D-Glu-L-Ala-allo-D-Ile-L-Leu-));
BQ-123 (Cyclo(-D-Trp-D-Asp-L-Pro-D-Val-L-Leu-)); TAK-044 (Cyclo(−4-oxo-4-(4-phenyl-1-piperazi-nyl)-L-2-aminobutanoyl-L-Asp-D-2-(2-thienyl)glycyl-L-Leu-D-Trp-D-Asp-) di-sodium salt); PD-142893 (N-Acetyl-(3,3-diphenyl-D-alanine)-L-Leu-L-Asp-L-Ile-L-Ile-L-Trp); PD-156252 (N-Acetyl-2-D-(10,11-dihydro-5B-dibenzo(a,d)cycloheptene-5-yl)-glycyl-L-Leu-L-Asp-L-Ile-N-methyl-L-Ile-L-Trp disodium salt); BQ-485 (Perhydroazepin-1-ylcarbonyl-L-leucyl-D-tryptophyl-D-tryptophan); Cochinmicin I or Myricerone caffeic acid ester.

Examples for non-peptidic ET receptor antagonists are:

Sitaxsentan (N-(4-Chloro-3-methylisoxazol-5-yl)-2-(2-(6-me-thyl-3,4-methylendioxy-1-yl)acetyl)thiophen-3-sulfonamide); TBC-3214 (N-(2-acetyl-4,6-dimethylphenyl)-3-{[(4-chloro-3-methyl-5-isoxazolyl)amino]sulfonyl}-2-thiophenecarboxamide);
TBC-3711; SB-209670 ((1S,2R,3S) 1-(3,4-methylendioxyphenyl)-3-(2-(carboxymethoxy)-4-methoxyphenyl)-5(prop-1-yloxy)indan-2-carboxylic acid); Bosentan (4-tert-Butyl-N-(6-(2-hydroxyethoxy)-5-(2-methoxyphenoxy)(2,2′-bipyrimidin)-4-yl)benzene-sulfonamide); PD-156707 (2-(3,4-Methylendioxyphenyl)-4-(4-methoxyphenyl)-4-oxo-3-(3,4,5-trimethoxybenzyl)-but-2-ene acid sodium salt); L-749329 (4-(2-(4-Isopropylphenylsulfonamido)-1-(3,4-methylenedioxyphenyl)-2-oxoethoxy)-3-propylbenzoic acid); L-754142 (4-(2-(4-Isopropylphenylsulfonamido)-1-(3,4-methylendioxyphenyl)-2-oxoethoxy)-3-propylbenzoic acid dipotassium salt); Enra-sentan ((1S,2R,3S) 1-(3,4-Methylendioxyphenyl)-3-(2-(2-hydroxyethoxy)-4-methoxyphenyl)-5(prop-1-yloxy)indan-2-carboxylic acid); A-127722 (trans-trans-2-(4-methoxyphenyl)-4-(3,4-methylendioxyphenyl)-1-(2-(N,N-dibutylamino)-2-oxoethyl)-pyrrolidine-3-carboxylic acid); Abtrasentan (ABT-627 ([2S-(2a,3b,4a)]-2-(4-methoxy-phenyl)-4-(3,4-methylendioxyphenyl)-1-(2-(N,N-dibutylamino)-2-oxoethyl)-pyrrolidine-3-carboxylic acid)); EMD-94246 (N-(2,1,3-Benzothiadiazol-5-yl)-5-(dimethylamino)naphthalin-1-sulfonamide potassium salt); ZD-1611 (3-(4-(3-(N-(3-Me-thoxy-5-methylpyrazin-2-yl)sulfamoyl)pyridin-2-yl)phenyl)-2,2-di-methylpropionic acid); K-8794 (N-(2,6-dimethylphenyl)-3-(6-(4-t-butylphenylsulfonylamino)-5-(2-methoxyphenoxy)-2-(2-pyrimidinyl)-4-pyrimidinyloxy) propionamide); A-182086 ((2a,3b,4a)-2-(3-Fluor-4-methoxyphenyl)-4-(3,4-methylendioxyphenyl)-1-(2-(pentylsulfonyl)propyl-amino)ethyl-pyrrolidine-3-carboxylic acid); PD-163070 ((2Z)-2-(1,3-benzodioxol-5-yl)-3-(3-(dimethylamino)benzyl]-4-(4-methoxyphenyl)-4-oxo-2-butenoic acid sodiumsalt); PD-166557 ((2Z)-3-(3-aminobenzyl)-2-(1,3-benzodioxol-5-yl)-4-(4-methoxyphenyl)-4-oxo-2-butenoic acid sodium salt); Ro-61-1790 (N-{6-(2-hydroxyethoxy)-5-(2-methoxyphenoxy)-2-(2-(1H-tetraazol-5-yl)-4-pyridinyl]-4-pyrimidinyl}-5-methyl-2-pyridinesulfonamide disodium salt BMS-193884 (N-(3,4-dimethyl-5-isoxazolyl)-4′-(1,3-oxazol-2-yl)[1,1′-biphenyl]-2-sulfonamide); BMS-207940; SB-209598 (3-[2-(carboxymethoxy)-4-methoxyphenyl]-1-[(6-chloro-1,3-benzo-dioxol-5-yl)methyl]-1H-indole-2-carboxylic acid); SB-209834 ((2E)-2-(1,3-benzodioxol-5-ylmethyl)-3-(2-butyl-1-methyl-1H-imidazol-5-yl)-3-(2-(carboxymethoxy)-4-methoxyphenyl]-2-propenoic acid); A-206377 ((2S,3R,4S)-4-(1,3-benzodioxol-5-yl)-1-(2-(dibutylamino)-2-oxoethyl 1-2-(2-(2-oxo-1-pyrrolidinyl)ethyl]-3-pyrrolidinecarboxylic acid); EMD-122801 ((2Z)-2-(2,1,3-benzothiadiazol-5-yl)-4-(4-methoxyphenyl)-4-oxo-3-(3,4,5-trimethoxybenzyl)-2-butenoic acid sodium salt); Tezosentan (N-{6-(2-hydroxyethoxy)-5-(2-methoxyphenoxy)-2-(2-(1H-tetraazol-5-yl)-4-pyridinyl]-4-pyrimidinyl-5-isopropyl-2-pyridinesulfonamide disodium salt); AC-61-0612(N-(6-(2-hydroxyethoxy)-5-(2-methoxyphenoxy)-2-(2-(1H-tetraazol-5-yl)-4-pyridinyl]-4-pyrimidinyl)-5-isopropyl-2-pyridinesulfonamide); T-0201 (N-(6-(2-[(5-bromo-2-pyrimidinyl)oxy]ethoxy}-5-(4-methylphenyl)-4-pyrimidinyl]-4-(2-hydroxy-1,1-dimethylethyl)benzenesulfonamide sodiumsalt); J-104132 ((5S,6R,7R)-5-(1,3-benzodioxol-5-yl)-2-butyl-7-(2-((2S)-2-carboxypropyl 1-4-methoxyphenyl}-6,7-dihydro-5H-cyclopenta[b]pyridine-6-carboxylic acid) and compounds of the general formula I:

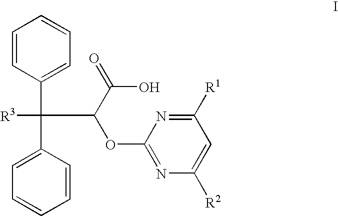

wherein R¹, R² and R³ are:

### R¹ C₁-C₄-Alkyl, C₁-C₄-Alkoxy;

### R² C₂-C₄-Alkyl, C₁-C₄-Alkoxy;

R³ C₁-C₈-Alkyl which may carry a phenyl which may carry up to 2 identical or different C₁-C₄-Alkoxy radicals.

Preferred compounds of the formula I are compounds, wherein R¹, R² and R³ are

### R¹ C₁-C₂-Alkyl, C₁-C₂-Alkoxy;

### R² C₁-C₂-Alkyl, C₁-C₂-Alkoxy;

R³ C₁-C₂-Alkyl which may carry a phenyl which may carry up to 2 identical or different C₁-C₂-Alkoxy radicals.

Orally available ET-receptor antagonists are listed, for example, in Douglas, S. A., Trends in Pharmacol. Sci., 18, 408-12, 1997, preferred are ABT-627, A-182086, PD163070, PD166557, Bosentan, TBC-11252 or ZD-1611.

Most preferred ET antagonists are (S)-2-(4,6-Dimethoxy-pyrimidin-2-yloxy)-3-methoxy-3,3-dipheny-propionic acid, (S)-2-(4,6-Dimethyl-pyrimidin-2-yloxy)-3-(2-(3,4-dimethoxy-phenyl)ethoxy)-3,3-diphenylpropionic acid, (S)-2-(4,6-Dimethyl-pyrimidin-2-yloxy)-3-methoxy-3,3-diphenyl-propionic acid or (S)-2-(4,6-Dimethyl-pyrimidin-2-yloxy)-3,3-diphenylbutanoic acid.

Preferred αᵥβ₃ integrin receptor antagonists within the scope of the invention are substances which show an IC50 value of 100 nM or 20 less for the inhibition of vitronectin binding to integrin αᵥβ₃ in an ELISA assay, which is, described for example in DE 19919218.9 (German application number).

Suitable αᵥβ₃ integrin receptor antagonists for the combination therapy of the invention are, in principle, all αᵥβ₃ integrin receptor antagonists, for example as described in Pitts et al.; J. Med. Chem. 2000, 43, 27-40; Batt et al., J. Med. Chem. 2000, 43, 41-51; Miller et al., Bioorg. Med. Chem. Lett. 9, 1999, 1807-1812; Keenan et al., Bioorg. Med. Chem. Lett. 9, 1999, 1801-1806; Rockwell et al., Bioorg. Med. Chem. Lett. 9, 1999, 937-942; Samanen et al., Current Pharm. Design 1997, 3, 545-584; Miller et al., J. Med. Chem. 2000, 43, 22-26; Hartmann and Duggan, Exp. Opin. Invest. Drugs 2000, 9 (6), 1281-1291; Miller et al., Drug Discovery Today 2000, 5 (9), 397-408; DE 19919218.9 (German application number), DE 19948269.1 (German application number), DE 19962998.6 (German application number), DE 10027514.1 (German application number), DE 10028575.9 (German application number), DE 10039998.3 (German application number), WO 9952879, WO 9835917, WO 0000486, WO 0017197, WO 0031067, WO 9843962, WO 9926945, WO 9950249, WO 9958162, WO 0000481, U.S. Pat. No. 6,056,958, WO 43787, WO 9637492, WO 9723480, WO 9733887, WO 9748395, WO 9748444, WO 9823608, U.S. Pat. No. 5,849,736, DE 19626701, EP 0796855A1, DE 19653645, DE 19653646, DE 19653647, EP 796855, EP 820988, EP 820991, EP 853084, EP 854145, U.S. Pat. No. 5,990,145, WO 9915506, WO 9915507, WO9932457, WO 9937621, WO 9959992, EP 928790, EP 928793, U.S. Pat. No. 6,001,855, WO 00024724, WO 9825892, WO 9965944, WO 0048603, WO 9938849, WO 9952872, DE 19534016, DE 19548709, DE 19653036, DE 19654483, DE 19705450, DE 1971300, DE 19725368, DE 19842415, DE 19850131, EP 683173, EP 710657, EP 741133, EP 771 818, WO 9714716, WO 9723451, WO 9738009, WO 9744333, WO 9800395, WO 9818764, WO 9827112, WO 9835949, WO 9901472, WO 9910371, WO 9931126, WO 0003973, WO 0026212, WO 9532710, WO 9726250, WO 9737655, WO 9808518, WO 9808840, WO 9818460, WO 9818461, WO 9831359, WO 9844797, WO 9846220, WO 9901472, WO 9930709, WO 9930713, WO 9931061, WO 9931099, WO 0006169, WO 0009503, U.S. Pat. No. 5,981,546, U.S. Pat. No. 6,017,925, U.S. Pat. No. 6,017,926, WO 9967230, WO 9734865, FR 2768734-A1, FR 2768736-A1, WO 0032578, U.S. Pat. No. 5,639,765, U.S. Pat. No. 5,681,820, U.S. Pat. No. 5,852,210, U.S. Pat. No. 5,972,986, U.S. Pat. No. 6,013,651, WO 9708145, WO 9736858, WO 9736859, WO 9736860, WO 9736861, WO 9736862, WO 9944985, WO 9944994, WO 9951638, WO 9952896, WO 0009143, WO 0038665, WO 0038715, WO 0038719, WO 0038786, WO 9600574, WO 9600730, WO 9606087, WO 9626190, WO 9701540, WO 9724119, WO 9724122, WO 9724124, WO 9724336, WO 9814192, WO 9815278, WO 9829561, WO 9830542, WO 9840488, WO 9905107, WO 9906049, WO 9911626, WO 9915170, WO 9915178, WO 9915508, WO 9945927, WO 0007544, WO 0033838 or WO 9933798, particularly, the following proteins, peptidic and nonpeptidic compounds.

Proteins and peptidic αᵥβ₃ integrin receptor antagonists:

LM 609 (vitaxin, Pharmaprojects), abciximab (c7E3 Fab, Reopro®, Pharmaprojects), Peptides and peptidomimetics of Arg-Gly-Asp and derivatives thereof like:
cyclo(RGDfV), As-Pen-RGDC-OH, cyclo[RGD-Mamb-P], XJ 735 (cyclo[R-G-D-Mamb-A]), XK 002 (cyclo[(NMe)R-G-D (2-amino-1,3-thiazol-4-yl-acetic acid)-V]), DMP 728 (cyclo[[(NMe)R-G-D-Mamb-Dabu]), SK+F 107260

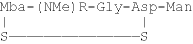

EMD 121974 (cyclo[R-G-D-f-(NMe)V]) and any other RGD containing peptides.

Non-peptidic αᵥβ₃ integrin receptor antagonists:

(2R)-2-[((2R)-2-{3-[(3-{[amino(imino)methyl]amino}propanoyl)amino]phenyl}-3-carboxy propanoyl)amino]-3-methylbutanoic acid, 3-[8-(2-{(amino(imino)methyl]amino}ethyl)-1-ben-zyl-2-oxo-1,2,3,5-tetrahydro-4H-1,4-benzodiazepin-4-yl]propanoic acid, 2,3-dihydroxypropyl 2-{[(benzyloxy)carbo-nyl]amino}-4-({9,10-dimethoxy-4-[(E)-2-(1,4,5,6-tetrahydropyrimidin-2-yl)hydrazono]-1,2,3,3a,4,5,6,10b-octahydrobenzo {e}azulen-8-yl}oxy)butanoate, (2S)-2-{((benzyloxy)carbonyl]amino}-3-[({(4S)-4-(3-(4,5-dihydro-1H-imidazol-2-ylamino) propyl]-2,5-dioxoimidazolidin-1-yl}acetyl)amino]propanoic acid, L-7418415 ((2S)-2-[(phenylsulfonyl)amino]-3-(0-(2-(1,4,5,6-tetrahydropyrimidin-2-ylamino)ethoxy]benzoyl}amino)propanoic acid), (2S)-2-{[(4-isobutylphenyl)sulfonyl]amino}-3-[({5-[3-(pyridin-2-ylamino)propyl]-4,5-dihydroisoxazol-3-yl}carbonyl)amino)propanoic acid, (2S)-2-{[(benzyloxy)carbo-nyl]amino}-3-[({4-[4-(4,5-dihydro-1H-imidazol-2-ylamino)butanoyl]piperazin-1-yl}carbonyl)amino]propanoic acid, (2S)-2-{[(benzyloxy)carbonyl]amino}-3-[({4-[4-(4,5-dihydro-1H-imidazol-2-ylamino)propanoyl]piperazin-1-yl}carbonyl)amino]propanoic acid, SD-186 ((2S)-2-[(phenylsulfonyl)amino]-3-[{(8-(pyridin-2-ylamino)methyl}-1-oxa-2-aza-spiro[4.5]dec-2-en-3-yl]carbonyl)amino]propionic acid), SD-183 ((2S)-2-[(phenylsulfonyl)amino]-3-[({8-[(pyridin-2-ylamino)methyl]-1-oxa-2-azaspiro[4.5]dec-2-en-3-yl]carbonyl)-amino]propanoic acid, SD-983 ((2S)-2-{[(benzyl oxy)carbonyl]amino}-3-[({3-(3-(4,5-dihydro-1H-imidazol-2-ylamino)propoxy] isoxazol-5-yl}carbonyl)amino]propanoic acid), XT-199 ((2S)-3-{[({3-(3-(4,5-dihydro-1H-imidazol-2-ylamino)propoxy]isoxazol-5-yl}carbonyl)amino]-2-[(phenylsulfonyl)amino[propanoic acid), SG-545 (Methyl (2S)-2-{[(benzy-loxy)carbonyl]amino}-3-[({3-(3-(4,5-dihydro-1H-imidazol-2-ylamino)propoxy]isoxazol-5-yl}carbonyl)amino]propanoic acid), SM 256 ((2S)-3-[({1-[3-(1H-imidazol-2-ylamino)propyl]-1H-indazol-5-yl}carbonyl)amino]-2-[(mesitylsulfonyl)amino] propanoic acid), SD-836 (Pharmaprojects), SD-7784 (Pharmaprojects), SD-7783 (Pharmaprojects), S-137 (N-({[1-(4-{[amino(imino)methyl]amino}butyl)vinyl]amino}acetyl)-3-pyridin-3-yl-beta-alanine), S-787 (Seattle et al.; 21^{st} Ann. Meet. Amer. Soc. Bone Mineral Res., Sep. 30-Apr. 10, 1999; SU 410), S 448 (N-{[(3-{[amino(imino)methyl]amino}benzoyl)amino]acetyl}-3-phenyl-β-alanine), SC 68448 (N-{[(3-{[amino(imino)methyl]amino}benzoyl)amino]acetyl}-3-(3,5-dichlorophenyl)-β-alanine), SC 56631 (N-{[(5-{[amino(imino)methyl]amino}pentanoyl)amino]acetyl)-3-pyridin-3yl-β-alanine), SC 69000 (4-[(3-{[amino(imino)methyl]amino}benzoyl)amino]-N-(isobutoxycarbonyl)phenylalanine), SC-65811 (N-{[(3-{[(benzylamino)carbonyl]amino}benzoyl)amino]acetyl}-3-pyridin-3-yl-b-alanine), SB 223245 (((2S)-7-{[(1H-benzimidazol-2-ylmethyl)(methyl)amino]carbonyl}-4-methyl-3-oxo-2,3,4,5-tetrahydro-1H-1,4-benzodiazepin-2-yl)acetic acid), SB 265123 ([(10S)-3-(3-(pyridin-2yl-amino)propoxy)-10,11-dihydro-5H-dibenzo[a,d]-cyclohepten-10yl)acetic acid), SB 267268 ([(4S)-3-oxo-8-(3-(pyridin-2-ylamino)propoxy]-2-(2,2,2-trifluoroethyl)-2,3,4,5-tetrahydro-1H-2-benzazepin-4-yl]acetic acid), SB 273005 (Lark et al.; 21^{st} Ann. Meet. Amer. Soc. Bone Mineral Res., Sep. 30-Apr. 10, 1999; SU201), CP-4632((2S)-3-[(3-fluoro-4-(4-(1,4,5,6-tetrahydropyrimidin-2-ylamino)piperidin-lyl]benzoyflamino]-2-[(phenylsulfonyl)amino]propanoic acid), (2S)-3-({3-chloro-4-[4-(1,4,5,6-tetrahydropyrimidin-2-yl)piperidin-1-yl]benzoyl}amino)-2-[(phenylsulfo-nyl)amino]propanoic acid), SH306 (2S)-2-[(mesitylsulfonyl)amino]-3-[({1-[3-(pyridin-2-ylamino)propyl]-1H-indazol-5-yl}carbonyl)amino]propanoic acid, SB 273005 (Lark et al.; 21st Ann. Meet. Amer. Soc. Bone Mineral Res., Sep. 30-Apr. 10, 1999; SU201) [(4S)-8-{2-[6-(Methylamino)pyridin-2-yl]ethoxy]-3-oxo-2-(2,2,2-trifluoroethyl)-2,3,4,5-tetrahydro-1H-2-benzazepin-4-yl]acetic acid, SC 72115 (3-(5-bromo-3-chloro-2-hydroxyphenyl)-N-({[3-(4,5-dihydro-1H-imidazol-2-ylamino)benzoyl]amino}acetyl)-beta-alanine).

Preferred are non-peptidic antagonists, particularly those which are orally available and αᵥβ₃ integrin receptor antagonists selected from the group:

LM 609 (vitaxin), EMD 121974 (cyclo[R-G-D-f-(NMe)V]), L-7418415 ((2S)-2-[(phenylsulfonyl)amino]-3-({4-[2-(1,4,5,6-tetrahydropyrimidin-2-ylamino)ethoxy]benzoyl}amino)propanoic acid), SB 265123 ([(10S)-3-[3-(pyridin-2yl-amino)propoxy]-10,11-dihydro-5H-dibenzo[a,d]-cyclohepten-IOyllacetic acid), SB 267268 ([(4S)-3-oxo-8-[3-(pyridin-2-ylamino)propoxy]-2-(2,2,2-trifluoroethyl)-2,3,4,5-tetrahydro-1H-2-benzazepin-4-yl]acetic acid), SB 273005 (Lark et al.; 21^{st} Ann. Meet. Amer. Soc. Bone Mineral Res., Sep. 30-Apr. 10, 1999; SU201), SC 68448 (N-([(3-{[amino(imino)methyl]amino}benzoyl)amino]lacetyl}-3-(3,5-dichlorophenyl)-β-ala-nine), SC 69000 (4-[(3-{[amino(imino)methyl]amino}benzoyl)amino]-N-(isobutoxycarbonyl)phenylalanine and SC-65811(N-{[(3-{[(benzylamino)carbonyl]amino}benzoyl)amino]acetyl}-3-pyridin-3-yl-b-alanine). All mentioned compounds can also be applied as prodrugs. Prodrugs are substances which metabolise in vivo to the active compound. Examples for such metabolism are first pass metabolism (e.g. esters to free acids or carboxylates).

All mentioned compounds may be administered as such or in the form of their salts with physiologically tolerated acids or bases

Preferred combinations of an endothelin blocker with an αᵥβ₃ integrin receptor antagonist are selected from the preferred endothelin blockers and the preferred αᵥβ₃ integrin receptor antagonists.

In a further preferred embodiment, orally available ET antagonists are combined with orally available αᵥβ₃ integrin receptor antagonists. “Orally available” means at least 10%, preferred 20% and more preferred 30% for ET antagonists and 30%, preferred 50% and more preferred 70% for αᵥβ₃ integrin receptor antagonist. The oral availability is for the purpose of this invention defined as measured in dogs as described in WO 9806740. The endothelin blocker in combination with the αᵥβ₃ integrin receptor antagonist may be adminstered together in a pharmaceutical composition or simultaneous via separate ways or separate or temporal graduated.

Therefore, the invention further relates to a pharmaceutical composition, comprising an endothelin blocker and an α_{vβ}₃ integrin receptor antagonist.

This composition can be used as a medicament, particularly for curing or preventing cardiovascular disorders, such as atherosclerosis, restenosis after vessel injury or revascularisation treatment, angioplasty (neointima formation, smooth muscle cell migration and proliferation), myokard infarkt or heart failure.

In a preferred embodiment, the composition is used for the treatment or prevention of restenosis after vessel injury or revascularisation treatment.

The compounds of the invention can be administered orally or parenterally in a conventionally way (subcutaneously, intravenously, intramusculary, intraperitoneally, rectally). Administration can also take place with vapours or sprays through the nasopharyngeal space. Oral administration is preferred. The dosage depends on age, condition and weight of the patient and on the mode of administration. The two compounds can be formulated in a single pharmaceutical form or in separate pharmaceutical forms. Administration can be given in several single doses (e.g. 2 to 4) or once or twice a day as depot form.

The weight ratio of αᵥβ₃ integrin receptor antagonist to endothelin blocker conveniently is in the range of 1:100 to 100:1 preferably 1:10 to 10:1. Advantageously, the dosage to be administered by means of a combination per day and kg amounts to 0,05 to 2 mg of an αᵥβ₃ integrin receptor antagonist and 0,1 to 50 mg of an endothelin blocker. In general, the total amount of an αᵥβ₃ integrin receptor antagonist and an endothelin blocker to be administered daily amounts per kg to a maximum of 50 mg. When a hydrate or a pharmaceutically usable salt is used, then the above values are to be appropriately adjusted.

The compounds can be used individually or together in conventional solid or liquid pharmaceutical forms, e.g. as uncoated or (film-)coated tablets, capsules, powders, granules, suppositories, solutions, ointments, creams or sprays. These are produced in a conventional way. In these, the active substances can be processed with conventional pharmaceutical aids such as tablet binders, fillers, preservatives, tablet disintegrants, flow regulators, plasticizers, wetting agents, dispersants, emulsifiers, solvents, release slowing agents, antioxidants and/or propellant gases (cf H. Sucker et al. Pharmaceutische Technologie, Thieme Verlag, Stuttgart, 1978). The administration form obtained in this way normally comprises the active substance in an amount of from 0.1% to 99% by weight.

Treatment of a patient with a cardiovascular disease by a combination, composition and method according to the present invention may include concomitant use of further adjunctive agents, such as antiplatelet agents, e.g., aspirin, and anticoagulant agents, e.g., heparin or low molecular weight heparin, or other drugs, e.g., b-blockers, angiotensin converting enzyme inhibitors, agents against reperfusion injury and others.

Subject of the present invention are also pharmaceutical compositions, comprising αᵥβ₃ integrin receptor antagonist in an appropriate container and an endothelin blocker in a separate container to be used according to the above-mentioned administration regiments.

Pharmaceutical packaging units prepared in accordance with the present invention may consist of an appropriate administration form comprising the αᵥβ₃ integrin receptor antagonist, and an appropriate packaging unit comprising the endothelin blocker. The two active compounds are preferrably present in the packaging unit in two different containers, e.g. tablets. However, depending on the type of active compound, it may also be possible to provide both compounds in a single dosage form. Further, the pharmaceutical packaging units comprise instructions, for example in the form of a package leaflet prescribed for medicaments from which it follows that the administration of a therapeutically active amount of the αᵥβ₃ integrin receptor antagonist advantageously takes place in combination with administration of an endothelin blocker.

If applied separately, the administration of the endothelin blocker takes places before, simultaneously or after the administration of the αᵥβ₃ integrin receptor antagonist. Information regarding the manner of use can either be given in the information leaflet or as a packing overprint on the medical preparation which can be bought together with medicinal preparations which comprise αᵥβ₃ integrin receptor antagonists. On the one hand, pharmaceutical packaging units comprising only appropriate administration forms of the αᵥβ₃ integrin receptor antagonists can comprise such information e.g. in the form of package leaflets, wherein the combined administration together with endothelin blockers according to the present invention is mentioned. On the other hand, pharmaceutical packaging units comprising only endothelin blockers can comprise such information wherein the combined administration together with αᵥβ₃ integrin receptor antagonists and the use according to the present invention is mentioned. A third alternative would be to provide pharmaceutical packaging units comprising an αᵥβ₃ integrin receptor antagonist, endothelin blocker and an appropriate information about the combined use of both, e.g. the usual package leaflet.

Therefore, the invention further relates to a pharmaceutical trade package, comprising as pharmaceutical agent an endothelin blocker or/and an αᵥβ₃ integrin receptor antagonist together with an instruction for use of this pharmaceutical agents in combination for simultaneous, separate, or temporal graduated application for the treatment or prevention of diseases.

Appropriate directions of use of the above-mentioned pharmaceutical agents are essential for commercialization of such pharmaceutical packages, comprising either the αᵥβ₃ integrin receptor antagonist, endothelin blocker or a combination thereof.

Commercialization of appropriate pharmaceuticals by pharmaceutical companies is only possible when prior approval of such pharmaceutical agents and the respective administration regimens is achieved by the respective national Health Authorities, such as the FDA in the US or the CPMP Authority in Europe.

This includes but is not limited to performing clinical trials according to well-established procedures under the supervision of said pharmaceutical company which later on intends to commercialize such pharmaceutical agents. This also includes filing of appropriate documentation about the results of such clinical trials with the respective Health Authority in order to get marketing approval. The approval is in many cases restricted to certain administration protocols or regimens which have to be included in printed form in the accompanying information leaflet prescribed for medicaments.

### EXAMPLES

### Example 1

### Integrin αᵥβ₃ (Human)/Vitronectin (Human) ELISA

96-well plates (Costar, cat #3369) were coated overnight at 4° C. with 100 μl/well integrin αᵥβ₃ (5 μg/1 ml) from human placenta in 50 mmol/l NaHCO₃ (pH 9.2). After (3×) washing with 0.05% Tween 20 in PBS, 50 μl of test buffer (0.1% skimmed milk powder in 50 mmol/1 Tris, 1 mmol/l CaCl₂, 1 mmol/l MgCl₂, 10 μmol/l MnCl₂, 100 mmol/l NaCl, 0.2% Tween 20) were pipetted into each well followed by 1 μl DMSO (control) or 1 μl αᵥβ₃ integrin receptor antagonist solution (1 mmol/l to obtain a final test concentration of 10 μmol/l) and by 50 μl vitronectin solution (2 μg/ml from human plasma. The wells were incubated for 2 h at room temperature and then washed again three times with 0.05% Tween 20 in PBS. Bound vitronectin was detected by incubation with 100 μl of peroxidase-coupled anti-vitronectin antibodies (0.5 μg/ml) in buffer containing 0.2% Tween 20 and 0.1% milk powder for 2 h at room temperature. After three washing steps with 0.05% Tween 20 in PBS, TMB solution 100 μl/well was added and incubated for 40 seconds at 37° C. The reaction was stopped by addition of 100 μl/well 2N H₂SO₄. Finally the absorbance as a measure for bound vitronectin was measured in a microplate photometer at 450 nm.

### Example 2

### Cellular Adhesion Assay (ELISA Technique)

24-well plates were coated overnight at 4° C. with human vitronectin 50 ng/well in 50 mmol/l NaHCO₃ pH 9.2. After washing with CHO—S—SFMII medium the wells were incubated for 1 h at 37° C. with recombinant CHO αᵥβ₃ expressing cells (subtype avb3-A: clone X, sub-type αᵥβ₃-B: clone 5, subtype avb3-C: clone 18) in CHO—S—SFMII medium (Gibco 12052-015) at a concentration of 1.10⁶ cells/ml with αᵥβ₃ integrin receptor antagonist added to obtain final concentrations between 0.1 nmol/l and 10 μmol/l for 2-3 h at 37° C. Plates then again were washed four times with CHO—S—SFMII medium. XTT (Roche 1465015) 500 μl/well in CHO—S—SFMII medium was added and incubated for 2-5 h at 37° C. Finally the absorbance as a measure for the count of adhesive cells vas measured in a photometer at 450 nm.

### Example 3

### BASMC Migration Assay

The migration of the primary human aortic SMC's was performed using 24-well Transwell cell culture chambers with an 8 μm pore size polycarbonate membrane (Costar3422). The lower surface of the filter was coated with vitronectin or osteopontin by incubation with 600 μl DMEM/0.2% albumin/±integrin αᵥβ₃-ligand (10 μg/ml); 3 hrs 37° C./5% CO₂. HASMC (Cascade Biologics; C-007-5C) were suspended in DMEM and 100 μl were placed in the upper compartment of the chamber (250-300000 cells/well) with or without αᵥβ₃ integrin receptor antagonist at various concentrations. The incubation was carried out at 37° C./5% CO₂ for 24 hours. The nonmigrated cells on the upper surface of the filter were removed by washing with PBS. The filters were transferred to a new 24-well plate and the lower compartments were incubated with 400 μl DMEM and 200 μl XTT (Roche 1465015) at 37° C./5% CO₂. After 24 hours the absorbance of 100 μl was measured at 450 nm.

### Example 4

### huCASMC Proliferation Assay

Cryopreserved primary human coronary artery smooth muscle cells (huCASMCs) were purchased from Clonetics® Cell Discovery Systems (catalog no. CC-2583). Cells were subcultured in T-75 flasks using smooth muscle cell growth medium-2 (SmGM-2) supplemented with 0.5 ng/ml recombinant human epidermal growth factor (hEGF), 5% foetal bovine serum, 5 μg/ml insulin, 50 μg/ml Gentamicin/50 ng/ml Amphotericin-B and human fibroblast growth factor-B (hFGFB) 2 ng/ml (Clonetics® Cell cat. no. CC-3182). For the proliferation assay cells between passage 5-8 were seeded at 5000 cells/well in 96 well plates in a volume of 0.1 ml and allowed to attach for 24 hours. Thereafter proliferating cells were incubated with an endothelin blocker and/or an αᵥβ₃ integrin receptor antagonist each at a concentration of 10−⁷M for further 24 hrs. Cell proliferation was quantitated in quadruplicates 24 hrs later using a colorimetric cell proliferation ELISA (BrdU, Roche Diagnostics, cat. no. 1 647 229).

### In Vivo Study

### Example 5

### Pig Model of Coronary Artery Restenosis

The pig coronary restenosis models is acknowledged as the only preclinical animal model with predictive value for the human pathology of restenosis. The findings show that the combination of ET receptor antagonists and αᵥβ₃ integrin receptor antagonists represent a more effective means of preventing restenosis than treatment with either drug alone. In fact, given the higher predictive value of the pig restenosis model, the in vitro results suggest effective prevention of human restenosis with combinations of ET receptor antagonists and αᵥβ₃ integrin receptor antagonists only, rather than monotherapy.

The study was performed in land race pigs applying PTCA only or PTCA plus implantation of a stent in the LAD according to standard clinical protocols 5 minutes prior to balloon inflation an ET_{A} receptor antagonist an αᵥβ₃ integrin receptor antagonist or a combination thereof was administered intravenously and after recovery from anaesthesia the animals received the compound/s orally, subcutaneously or by continuous infusion for 4 or 12 weeks. At the end of the experiments the LAD was excised and frozen to allow receptor distribution studies and histological examination with assessment of intimal hyperplasia after angioplasty by determination of residual lumen and the neointima/media ratio.

### Detailed in Vivo Method:

| | |
|---|---|
| Animals: | Species: domestic pig |
| Sex: | male and female |
| Age: | 8 to 9 weeks |
| Weight: | 20 to 30 kg |

Breeder: Dortans, Schatthausen, Germany. The health status of the animals used is controlled by a veterinary surgeon. Before start of the experiment, animals are acclimatized for at least 1 week. During this period the animals are trained to receive a small amount of food (mashed potatoes) before they are fed their standard maintenance diet (Ssniff Spezialdidten GmbH, Soest), so that the drug containing mixture is quantitatively eaten by each animal within a few minutes. Drinking water is available ad libitum.

### Study Design:

40 pigs of both sexes are allocated at random to one of the following treatment groups:

1. control
2. αᵥβ₃ integrin receptor antagonist: in a range between 0.05 and 5 mg/kg/d s.c. or as an iv infusion between 0,01 and 1 mg/kg/h.
3. ET_{A} receptor antagonist in a range between 0.1 and 50 mg/kg/d p.o.
4. ET_{A} receptor antagonist plus integrin αᵥβ₃ antagonist [in a ratio between 1:10 and 10:1 and a total doses range between 0.1 and 50 mg/kg/d p.o. or 5 mg/kg/d s.c. or 0,01 to 1 mg/kg/h infusion] administration: once daily between 07:00 and 9:00 a.m.subcutaneously or orally, by giving freshly prepared mashed potatoes mixed with a calculated amount of drug.

Duration of treatment: 4 or 12 weeks, beginning one day before angioplasty.

All animals will receive an i.v. bolus injection of a tenth of the oral dose just before the angioplasty

Experimental procedure: 24 h before surgical intervention, the animals are given 650 mg acetylsalicylic acid (ASS, Ratiopharm) and 30 mg nifedipine orally in addition to the test substance or placebo. For introduction and maintenance of general anaesthesia during angioplasty, the animals are given 2 mg/kg Stresnil® i.m. (Azaperon), followed by 4 mg/kg Metomidat® i.v. (Eypnodil, Janssen). Animals are intubated (RUsch Mikrolaryngealtubus, I.D.: 4.0 mm) and ventilated with 75% N₂0 and 25% O₂. An 8F sheath (Cordis, FI 33102-5700) is placed retrogradely in the right carotid artery. Adequate anticoagulation is achieved by intraarterial bolus injection of 7000 IU of unfractionated heparin (Thrombophob®, Nordmark, Uetersen). A standard PTCA guide catheter (Judkin left, powerbase 8F, ACS; mandrin=softguide, soft type, ACS) is then advanced via the aortic root into the left coronary artery under x-ray guidance. The balloon catheter (RX Elipse 0.014, ACS) is positioned in the first third of the left coronary artery using a 0.014″ PTCA guide wire (Galeo, Biotronic, Berlin). After X-ray control of its position, the balloon catheter is expanded by inflating the balloon twice to 10 atm for 30 sec. After deflating the balloon and after withdrawing it, an additional angiogram is made to verify the lesion.

Specimen collection: 28 or 92 days after balloon angioplasty the animals are anaesthetized as described above. Thereafter the animals receive a relaxant (Impretil®, hexacarbocholinbromide, 0.03 mg/kg i.v.), the chest is opened and the heart removed. The dilated coronary artery segments including the adjacent noninjured segments are then carefully dissected from the epicardial surface, transferred into PBS and tissue tek is injected into the artery. After freezing the artery is sectioned transversely into 4 mm pieces. 4 sections from each segment are used for analysis (determination of area media, intima and lumen) after staining with hematoxylin-eosin (HE).

The cross sectional area of each segment is determined with digital morphometry. Neointimal thickness is determined as difference of the residual lumen area from the total area within the internal elastica lamina, which is considered as the normal lumen area. An uninjured proximal and distal segment of 4 mm length each was used as a reference lumen.

Parameters measured: Incidence of acute events (acute myocardial infarction, death, cardiac arrhythmia, coronary artery spasm, arterial dissection, haematoma), neointimal-, lumen-, media-, and adventitial area and mean thickness to be determined in 4 mm intervals of the dilated area. Angiographic lumen diameter before and after balloon inflation and at the end of the 28 or 29 day treatment period.

Evaluation: Results are expressed as mean±SEM or as median. Comparisons between groups are made using Dunnett's test for unpaid-red samples. Correlations between injury score and neointima formation are used to compare the effect of different treatments.

The use of the combination of an endothelin blocker and an αᵥβ₃ integrin receptor antagonists achieves a reduction of restenosis significantly more pronounced then one of the two treatments alone at the given doses. The combination of an ET blocker and an αᵥβ₃ integrin receptor antagonists in doses too low to be effective alone is effective as a high mono-therapy with either agent and has less potential for side-effects than one principle alone.

1. Use of an endothelin blocker in combination with an αᵥβ₃ integrin receptor antagonist for the manufacture of medicaments for the treatment of prevention of diseases.

2. Use as claimed in claim 1 for the treatment or prevention of cardiovascular disorders.

3. Use as claimed in claim 2 for the treatment or prevention of restenosis after vessel injury or revascularisation treatment.

4. Pharmaceutical composition, comprising an endothelin blocker and an α₁β₃ integrin receptor antagonist.

5. Composition as claimed in claim 4 for use as a medicament.

6. Composition as claimed in claim 5 for curing cardiovascular disorders.

7. Use of a composition as claimed in claim 4 for the manufacture of a medicament for the treatment or prevention of diseases.

8. Use a claimed in claim 7 for the treatment or prevention of cardiovascular disorders.

9. Use as claimed in claim 8 for the treatment or prevention of restenosis after vessel injury or revascularisation treatment.

10. Trade package, comprising as pharmaceutical agent an endothelin blocker or/and an αᵥβ₃ integrin receptor antagonist together with an instruction for use of this pharmaceutical agents in combination for simultaneous, separate, or temporal graduated application for the treatment or prevention of diseases.
